(19) United States
(12) Patent Application Publication
(10) Pub. No.: US 2025/0078982 A1
(43) Pub. Date: Mar. 6, 2025

(54) System and methods for automatically generating cognitive exercises

(71) Applicant: Qnaptic, Inc., Palo Alto, CA (US)

(72) Inventors: Jeff Argast, Palo Alto, CA (US); Ashutosh Malaviya, San Jose, CA (US); Satish Menon, Sunnyvale, CA (US); Xin Wen, Sunnyvale, CA (US); Hallie Whitney Mass, San Jose, CA (US); Erik Weitzman, Redwood City, CA (US); Julia Sze Ling Gan, Fremont, CA (US)

(73) Assignee: Qnaptic, Inc., Palo Alto, CA (US)

(21) Appl. No.: 18/735,080

(22) Filed: Jun. 5, 2024

Related U.S. Application Data
(60) Provisional application No. 63/579,495, filed on Aug. 29, 2023.

(51) Int. Cl. G16H 20/70; G16H 50/20

(52) U.S. Cl. CPC G16H 20/70; G16H 50/20

(57) ABSTRACT

A computer system for automatically generating cognitive exercises includes a computer memory that can store a memory list comprising subjects that a user has difficulty to remember, wherein the computer memory can store personal data associated with the user, and computer processors in communication with the computer memory can execute a cognitive exercise computation agent and a large language model responsive to the cognitive exercise computation agent. The cognitive exercise computation agent can retrieve the memory list and the personal data associated with the user from the computer memory. The cognitive exercise computation agent can produce generated cognitive exercises using the large language model under constraints of the memory list and the personal data associated with the user.

### BACKGROUND OF THE INVENTION

The present application relates to computer technologies for personal wellness enhancement, and in particular, to technologies that can automatically generate exercises using artificial intelligence.

Many people have a need to improve their memories of words. People may forget more words due to aging, or medical conditions such as Alzheimer, aphasia, and dementia. Practicing word exercises can be helpful in enhancing people's memories and prepare people better for their daily lives.

One drawback of conventional technologies: exercise content becomes stale and repetitive, and users lose interest. Another drawback of conventional technologies: the exercises are generic and not specific to users' needs. Yet another drawback of conventional technologies is that passive memory is disconnected from active daily usages.

Moreover, conventional technologies have a limited set of words and exercises, which constrains the ability to customize exercises for a particular person. Conventional technologies cannot take an arbitrary word or phrase and generate exercises for the person to work on.

There is therefore a need for computer tools that can help enhance mental wellness with problems relevant to users and fresh and stimulating content.

### SUMMARY OF THE INVENTION

The presently disclosed system and method disclose innovative computer technologies aimed at assisting users in improving their memories. The systems and methods can automatically generate cognitive exercises to improve memory lapses caused by aging, stress, lack of sleep, or medical conditions like Alzheimer's, aphasia, and dementia.

The disclosed system and method provide personalized exercise content that is relevant to the users. The exercise content is tailored to the user's interests, life experiences, and personal details, making the exercises more relatable and engaging. The exercises can target the specific needs of users' skill development. Understanding of user's specific strengths and weaknesses allows the content to focus on developing the most relevant skills and abilities. The user's progress and difficulties are tracked. The exercise content can be adjusted to provide an appropriate level of challenge and complexity.

The styles of the exercises are also personalized to adapt exercise styles to user's preferences, exercise formats, and learning style to enhance motivation and engagement. The disclosed system can monitor user's interaction history to provide timely and appropriate interventions or feedback tailored to their specific needs during an exercise.

The disclosed system and method integrate AI algorithms to provide timely, targeted, and fresh content into speech therapy exercises with significantly enhanced personalization, relevance, and effectiveness of the therapy, which lead to improved engagement, better skill development, and more successful outcomes for the users. By leveraging AI and up-to-date information sources, the exercises can remain highly relevant and contextual, reflecting current events, trends, or situations that resonate with the user's life and interests. This heightened relevance can improve engagement and make the learning experience more meaningful. AI algorithms can also analyze the user's performance, preferences, and needs in real-time, enabling the system to dynamically generate or select exercises that are tailored and targeted to their specific areas of improvement. This targeted and customized approach can optimize the learning process and accelerate skill development. The AI-enabled exercises can also simulate real-world scenarios and situations, better preparing the user to apply their acquired speech and language skills in practical contexts outside of the therapy environment.

Furthermore, the disclosed system and method incorporate human-like qualities to encourage widespread user acceptance. By humanizing the system, making it relatable and attuned to human values and sensitivities, which encourages users' engagement with and adoption. This approach fosters a user-friendly environment, increasing the likelihood that people will embrace and use the software for their memory assistance needs.

In one general aspect, the present invention relates to a computer system for automatically generating cognitive exercises, which includes a computer memory that can store a memory list comprising subjects that a user has difficulty to remember, wherein the computer memory is configured to store personal data associated with the user, and one or more computer processors in communication with the computer memory can execute a cognitive exercise computation agent and a large language model responsive to the cognitive exercise computation agent, wherein the cognitive exercise computation agent can retrieve the memory list and the personal data associated with the user from the computer memory, wherein the cognitive exercise computation agent can produce generated cognitive exercises using the large language model under constraints of the memory list and the personal data associated with the user.

Implementations of the system may include one or more of the following. The computer memory can store a personal knowledge graph associated with the user, wherein the cognitive exercise computation agent can produce the generated cognitive exercises based on the personal knowledge graph using the large language model. The personal knowledge graph can include a user node representing the user, one or more first nodes in connection with the user node, representing relationships that the user has, and one or more second nodes in connection with the user node, representing interests and hobbies of the user. The computer memory can store exercise styles comprising concrete, focused, broad, stimulative, imaginative, exploratory, personalized, conversational, real-life situation, multimedia, multi-sensory, kinesthetic that involves physical movement or gestures, collaborative with other users, adaptive, progressive, or thematic around specific themes, or narratives, wherein the cognitive exercise computation agent can control the large language model to produce generated cognitive exercises further under constraint of the exercise styles stored in the computer memory. The cognitive exercise computation agent can include a cognitive exercise generator that can produce the generated cognitive exercises using the large language model under constraints of the memory list and the personal data associated with the user. The cognitive exercise computation agent comprises a cognitive exercise generation controller that can receive the memory list and the personal data associated with the user, wherein the cognitive exercise generation controller can control the cognitive exercise generator and the large language model to produce the generated cognitive exercises under constraints of the memory list and the personal data associated with the user. The cognitive exercise generation controller can receive user responses or feedback, wherein the cognitive exercise generation controller can steer the cognitive exercise generator and the large language model to produce improved cognitive exercises in response to user responses or feedback. The cognitive exercise generation controller can receive an example cognitive exercise, wherein the cognitive exercise generator can produce the generated cognitive exercises that mimic the example cognitive exercise. The cognitive exercise computation agent can include an image/video generator that can produce images or videos using the large language model under constraints of the memory list and the personal data associated with the user, wherein the images or videos can be incorporated into the generated cognitive exercises. The cognitive exercise computation agent can include a voice generation module that can produce a voice to be incorporated into the generated cognitive exercises. The cognitive exercise computation agent can include a multimodal module configured to receive user responses and feedback in one or more forms of voice, images, video, text, or touch. The memory list can include daily routine activities, healthcare needs, relationships, words, objects, experiences, events, interests, hobbies, occupational and professional terminologies, past experiences, professional activities, historical facts, literature, art, or music, or topics that are specific to user's needs. The computer memory can store a list of enhancement areas in which the user's memory needs to be improved, wherein the cognitive exercise computation agent can produce the generated cognitive exercises using the large language model further in accordance to enhancement areas associated with the user, wherein the enhancement areas can include motor-muscular-phonetics exercises, language syllable exercises, language exercises, cognitive exercises including convergence naming, divergence naming, or confrontation naming, daily activities, levels of memory loss, or general memory exercises for brain wellness.

In another general aspect, the present invention relates to a computer system for automatically generating cognitive exercises, which includes a computer memory that can store a memory list comprising subjects that a user has difficulty to remember, wherein the computer memory can store personal data associated with the user in which the user's memory needs to be improved; and one or more computer processors in communication with the computer memory, the one or more computer processors that can execute a cognitive exercise computation agent and a large language model responsive to the cognitive exercise computation agent, wherein the cognitive exercise computation agent comprises a cognitive exercise generation controller that can retrieve the memory list and the personal data associated with the user from the computer memory, wherein the cognitive exercise computation agent comprises a cognitive exercise generator, wherein the cognitive exercise generation controller can control the cognitive exercise generator and the large language model to produce the generated cognitive exercises under constraints of the memory list and the personal data associated with the user, wherein the cognitive exercise generation controller can receive user responses or feedback, wherein the cognitive exercise generation controller can steer the cognitive exercise generator and the large language model to produce improved cognitive exercises in response to user responses or feedback.

Implementations of the system may include one or more of the following. The computer memory can store a personal knowledge graph associated with the user, wherein the cognitive exercise computation agent can control the large language model to produce improved cognitive exercises based on the personal knowledge graph. The personal knowledge graph comprises a user node representing the user, one or more first nodes in connection with the user node, representing relationships that the user has, and one or more second nodes in connection with the user node, representing interests and hobbies of the user. The computer memory can store exercise styles comprising concrete, focused, broad, stimulative, imaginative, exploratory, personalized, conversational, real-life situation, multimedia, multi-sensory, kinesthetic that involves physical movement or gestures, collaborative with other users, adaptive, progressive, and thematic around specific themes, or narratives, wherein the cognitive exercise computation agent can control the large language model to produce generated cognitive exercises further under constraint of the exercise styles stored in the computer memory.

In another general aspect, the present invention relates to a computer-implemented method for automatically generating cognitive exercises, which includes storing personal data of a user in a computer memory, storing a memory list of subjects for cognitive enhancement for the user in the computer memory, generating content for cognitive exercises by a cognitive exercise computation agent, executed by one or more computer processors, using LLM and/or NLP tools based on the personal data and the memory list stored in the computer memory, generating cognitive exercises by the cognitive exercise computation agent using the LLM and/or the NLP tools, and presenting the cognitive exercises to the user by a computer device. Implementations of the system may include one or more of the following. The computer-implemented method can further include developing a personal knowledge graph for the user; and generating the content for cognitive exercises by the cognitive exercise computation agent using LLM and/or NLP tools further based on the personal knowledge graph. The computer-implemented method can further include selecting an exercise style for the user by the cognitive exercise computation agent, selecting an exercise type for the user by the cognitive exercise computation agent, and generating the cognitive exercises of the selected exercise type in the selected exercise style using LLM and/or NLP tools by the cognitive exercise computation agent. The computer-implemented method can further include receiving responses to cognitive exercises and feedback from the user, analyzing the responses and the feedback to the cognitive exercises, and steering generation of improved cognitive exercises by the cognitive exercise computation agent.

### BRIEF DESCRIPTION OF DRAWINGS

FIG. 1 is a system diagram of a computer system for automatically generating cognitive exercises in accordance with some embodiments of the present invention.

FIG. 2 is a detailed block diagram for a cognitive exercise computation agent compatible with the automated system in FIG. 1 in accordance with some embodiments of the present invention.

FIG. 3 is an exemplified knowledge graph for automatically generating cognitive exercises in accordance with some embodiments of the present invention.

FIG. 4 is a flowchart for automatically generating cognitive exercises in accordance with some embodiments of the present invention.

### DETAILED DESCRIPTION OF THE INVENTION

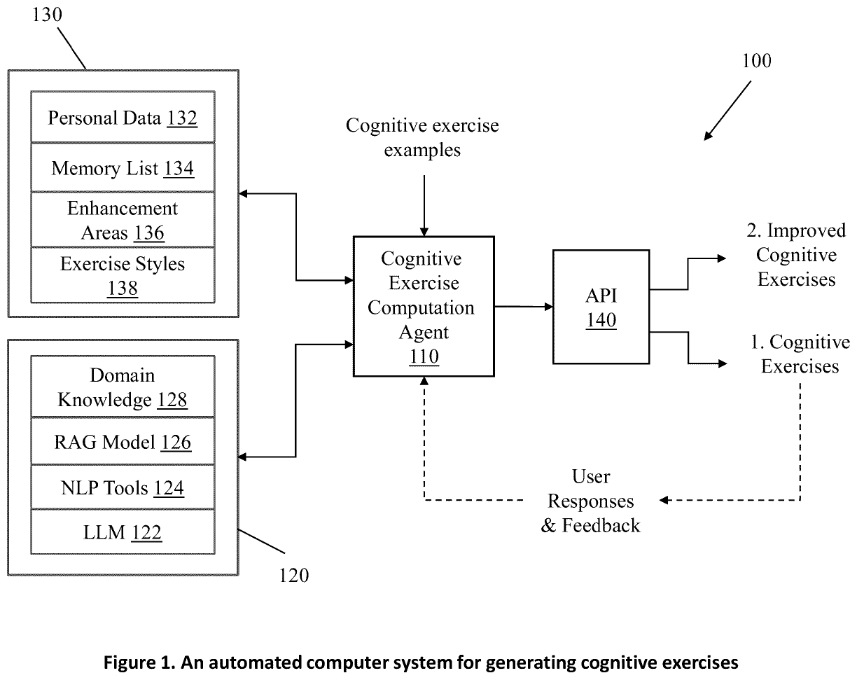

FIG. 1 shows a computer system 100 for automatically generating cognitive exercises. The computer system 100 includes one or more computer processors and computer memory. The computer system 100 includes a cognitive exercise computation agent 110, a language processing system 120, an individualized knowledge system 130 stored in the computer memory, and an application programming interface (API) 140, which are installed and executed on the one or more computer processors. The language processing system 120 can include one or more large language models (LLMs) 122 and/or one or more natural language processing (NLP) tools 124, a retrieval-augmented generation (RAG) module 126, and a domain knowledge database 128. The individualized knowledge system 130 can include personal data 132, a memory list 134, enhancement areas 136, and exercise styles 138.

The LLM 122 is a deep learning algorithm that can perform a variety of natural language processing tasks. The LLM 122 uses transformer models trained using large datasets. The NLP tools 124 uses machine learning to analyze text to uncover meaningful information and insight. In comparison to the LLM 122, the NLP tools 124 require less computation and data resources, which typically have faster processing rate and are less costly to operate. The RAG model 126 retrieves facts from an external knowledge base to ground the LLM 122 and the NLP tools 124 on the most accurate, up-to-date information and to give users insight into LLMs' generative process. The domain knowledge database 128 can include information related to memories, speech therapy, brain wellness, cognitive disorders, and aging. The information stored in the domain knowledge database 128 can include the symptom patterns and remedy methods for the memory loss of words, the retrieval of words, and the verbalization of the words. In some embodiments, domain knowledge can be represented in the format of knowledge graphs to better capture users' relationships, interests, and personal histories.

The individualized knowledge system 130 stores information specific to a user: words that the user often forgets during daily activities. The personal data 132 can include a user's important relationships such as family, friends, and caretakers, daily activities, the user's interests and hobbies, the user's life experiences such as upbringing, education and occupations, and travel experiences, places important to the user, notable events, and accomplishments in the user's life, etc. The personal data 132 can include user's unique circumstances, challenges, and life experiences. The personal data 132 can be built up during app initiation process or training sessions and updated during use sessions based on feedback from the user.

The memory list 134 includes subjects in which cognitive enhancement is needed for the user. The user may have difficulty remembering the subjects in the memory list 134. The subjects can include daily routine activities, healthcare needs, relationships, words, objects, experiences, events, interests, hobbies, occupational and professional terminologies, or details related to their past or current professions, historical facts, literature, art, music, and various other topics or domains that are relevant and challenging for the specific user's needs. By comprehensively capturing the subjects and areas where the user experiences memory difficulties, the system can design targeted exercises and interventions to strengthen their recall and retention abilities across a wide range of contexts. This approach ensures that the therapy addresses the user's unique memory challenges holistically, rather than being limited to a narrow set of domains. Additionally, the memory list 134 can be dynamically updated and refined based on the user's progress, newly identified areas of difficulty, or changing interests and life circumstances. This adaptability allows the therapy to evolve alongside the user's needs, providing a personalized and continuously relevant memory-enhancement experience. The memory list 134 can also include user's daily activities such as breakfast, coffee, tea, walk, exercises, playing games, meditation, lunch, nap, shower, going to bathroom, household chores, cooking, dinner, listening to podcast, watching video streaming, shopping, reading, listening to news.

Examples of the enhancement areas 136 can include motor-muscular-phonetics exercises, language syllable exercises, language exercises, cognitive exercises (convergence naming, divergence naming, confrontation naming, etc.), daily activities, levels of memory loss (e.g., mild, moderate, severe), and general memory exercises for brain wellness. Based on enhancement areas 136, the personal data 135, and the memory list 134, the generated cognitive exercises can include: “What are the names of your grandchildren?”, “What are the ages of your grandchildren?”, “Where do they go to school?”, “What are the interests and hobbies of each grandchild?”, “What are some recent major events or accomplishments in their lives?”, “Can you describe the last family gathering or vacation you had with them?”, “Who are your grandchildren's closest friends?”, “What are your grandchildren's favorite foods or restaurants?”, “Can you recall any funny or memorable stories involving your grandchildren?”, “What are your grandchildren's plans or goals for the future?”, “Can you name the teachers or coaches who work with your grandchildren?”, “What are some of the challenges or struggles your grandchildren are currently facing?”, “Can you describe the personality traits or characteristics of each grandchild?”, “What are some of the traditions or customs your family shares with your grandchildren?”, “Can you recall any significant milestones or achievements in your grandchildren's lives?” These questions and scenarios leverage the personal data 135 and memory list 134 to create exercises that are highly relevant, emotionally engaging, and tailored to the user's specific relationships, life experiences, and memory challenges. By incorporating familiar names, places, events, and details, the exercises become more meaningful and effective in enhancing the user's cognitive abilities and memory recall.

The exercise styles 138 can include a range of styles such as concrete, focused, broad, stimulative, imaginative, exploratory, personalized, conversational, real-life situation, multimedia, multi-sensory, kinesthetic that involves physical movement or gestures, collaborative with users, family and friends, or therapists, adaptive, progressive, and thematic around specific themes, or narratives. This diverse range of exercise styles allows the system to cater to different learning preferences, cognitive abilities, and therapeutic goals, ensuring an engaging and effective personalized experience for each user.

The individualized knowledge system 130 can continuously update and refine the personal data 132 and the memory list 134, ensuring that the exercises remain current and reflective of the user's evolving needs, interests, and life changes. This dynamic adaptation enables the therapy to remain highly personalized and effective throughout the user's journey, fostering progress and successful outcomes.

To generate cognitive exercises, the cognitive exercise computation agent 110 can receive examples of cognitive exercises such as a convergent exercise. The cognitive exercise computation agent 110 employs the LLM 122 and the NLP tools 124 to generate cognitive exercises mimicking the examples of cognitive exercises. The exercise example and the generated cognitive exercises can be in many different forms such as convergence naming, divergence naming, confrontation naming, filling blanks in a phrase, synonyms and antonyms, and subject-verb-object sentences. In convergence naming, for example, the exercise example may include giving a user “apple, pear, and banana” as the cue with “fruit” as the answer to the name of the group of the input words. The exercise can be presented visually, auditorily, or both. The user can respond by saying the answer or tapping on the correct answer. In one aspect, the content of the cognitive exercises generated by the cognitive exercise computation agent 110 employing the LLM 122 and the NLP tools 124 are based on the domain knowledge 128. The domain knowledge 128 can draw from established scientific literature and medical guidelines on cognitive impairments, such as: the Diagnostic and Statistical Manual of Mental Disorders (DSM-5) by the American Psychiatric Association, which provides diagnostic criteria and information on various cognitive disorders; Guidelines from the American Speech-Language-Hearing Association (ASHA) on cognitive-communication disorders and evidence-based practices for cognitive rehabilitation; research studies and meta-analyses published in peer-reviewed journals, such as the Journal of Speech, Language, and Hearing Research, Neuropsychological Rehabilitation, and the Journal of the International Neuropsychological Society, which examine the effectiveness of different cognitive interventions and exercise approaches; neuroscience literature on the underlying neural mechanisms of cognitive processes, such as attention, memory, executive functions, and language, to inform the design of targeted exercises; gerontology and geriatric medicine resources, such as the Handbook of the Neuroscience of Aging by Elsevier, which provide insights into age-related cognitive changes and appropriate interventions; clinical practice guidelines and recommendations from organizations like the Alzheimer's Association, the National Institute on Aging, and the American Academy of Neurology, which offer evidence-based strategies for cognitive rehabilitation in various conditions, such as dementia, stroke, and traumatic brain injury. By leveraging this scientific and medical knowledge, the cognitive exercise computation agent 110 can design exercises that are grounded in current research and tailored to specific cognitive deficiencies, maximizing the potential for effective rehabilitation and cognitive enhancement.

In some embodiments, examples of cognitive exercises are not received by the multimodal 210 for automatically generating cognitive exercises. Instead, the format of the cognitive exercises is provided by the world knowledge in the LLM 122 and NLP Tools 124. With the prompt or steering of cognitive exercise type, the LLM 122 and NLP Tools 124 can automatically generate exercises to mimic the specific types.

In another aspect, the content of the cognitive exercises generated by the cognitive exercise computation agent 110 employing the LLM 122 and the NLP tools 124 can incorporate personal data 132 to make it relevant to the specific user. The exercises can cover a diverse range of topics and interests tailored to different user profiles, allowing for the generation of highly personalized cognitive exercises across various domains. For example, for a pet owner of a fish or a dog, exercises can be automatically generated by the cognitive exercise computation agent 110 by giving the user a few names of a type of pet and askes the user to answer the category name of that type of pet: prompt to the user: Goldfish, Betta, Koi, with a correct answer: fish; prompt to the user: Koi, Carp, Goldfish, with a correct answer: fish; prompt to the user: Koi, Sakura, Ogon, with a correct answer: Koi varieties. The user can also be asked in exercises to list types of pet fish, popular pond fish, and varieties of Loi fish in different exercises.

In another example, if the user is known to be a sports lover, exercises can be automatically generated by the cognitive exercise computation agent 110 by giving the user a few names in a sports category and askes the user to answer the category name: prompt to the user: Patriots, Broncos, Seahawks, with a correct answer: NFL Teams; prompt to the user: Quarterback, Linebacker, Running Back, with a correct answer: Football Positions; prompt to the user: Holding, Offside, Pass Interference, with a correct answer: Football Penalties; prompt to the user: Hail Mary, Flea Flicker, Play Action Pass, with a correct answer: Football Plays. The user can also be asked in exercises to list names of NFL Teams, Football Positions, Football Penalties, and Football Plays.

In yet another example, if the user is known to be a runner or athlete, exercises can be automatically generated by the cognitive exercise computation agent 110 by giving the user prompt: Boston, New York, Chicago, London, etc., with a correct answer: Popular Marathon Races. The user can also be asked in exercises to list essential marathon training gear, strategies for hitting the wall and pushing through, and post-race recovery techniques.

In still another example, if the user is known to be a basketball lover, exercises can be automatically generated by the cognitive exercise computation agent 110 by giving the user prompt: crossover, spin move, Euro step, etc., with a correct answer: basketball moves. The user can also be asked in exercises to list names of NBA Teams, basketball legends and their accomplishments, and basketball terminology and slang.

In another example, if the user is known to be a Yoga/Tai Chi hobbyist, exercises are automatically generated by the cognitive exercise computation agent 110 by giving the user prompt: Yang, Chen, Wu, etc., with a correct answer: different styles of Tai Chi; and by giving the user prompt: Warrior Poses, Balancing Poses, etc., with a correct answer: Yoga Asanas. The user can also be asked in exercises to list the benefits of Tai Chi/Yoga practices, and mindfulness and breathing techniques.

In yet another aspect, the cognitive exercises generated by the cognitive exercise computation agent 110 employing the LLM 122 and the NLP tools 124 can specifically target areas in which the user needs to enhance memories, as indicated in the memory list 134. The exemplified exercise areas can include short-term memory exercises, long-term/episodic memory exercises, incidental/contextual memory exercises, semantic/factual memory exercises, semantic/factual memory exercises, and procedural memory exercises. In short-term memory exercises, for example, a user can be prompted to remember the starting lineup for the last NBA game the user watched (Basketball), to recall the ingredients you need to buy for your next meal prep (General), or to memorize and repeat back a sequence of yoga poses (Yoga/Tai Chi). In Long-term memory exercises, for example, a user can be prompted to describe in detail the user's experience running the Big Sur or Napa Marathon last time (Marathon), to recount a memorable family gathering or tradition from user's childhood (General), and to recall the key events and figures from the Civil Rights Movement (Social Justice). In incidental/contextual memory exercise, the user can be prompted to name the companies and logos you saw on billboards during your drive today (General), to identify the player numbers and positions of defensive players in a football highlight clip (Football), or to recall background details from the last documentary you watched on financial markets (Finance). In semantic/factual memory exercises, the user can be prompted to list the different investment strategies and their definitions (Finance), to explain the meaning and significance of common Buddhist terminology (Yoga/Tai Chi), or to describe the various ranks and roles within a Jehovah's Witness congregation (Jehovah's Witness). In procedural memory exercises, the user can be prompted to demonstrate the proper technique for a basketball layup (Basketball), to walk through the steps of performing a particular yoga sequence (Yoga/Tai Chi), or to outline the process for calculating a company's price-to-earnings ratio (Finance). By tailoring the exercises to target different types of memory (short-term, long-term, incidental, semantic, procedural) and drawing from personalized themes and interests, the cognitive exercise computation agent 110 can provide a comprehensive and engaging approach to enhancing the user's memory abilities across various contexts and domains.

Moreover, the cognitive exercises generated by the cognitive exercise computation agent 110 employing the LLM 122 and the NLP tools 124 can focus on the enhancement areas 136 stored in the individualized knowledge system 130. For instance, if the enhancement area 136 indicates a need for language exercises, the agent can generate exercises that target vocabulary building, sentence construction, or conversational fluency. If the enhancement area 136 specifies motor-muscular-phonetics exercises, the agent can design exercises that involve articulation drills, tongue twisters, or lip and jaw exercises to improve speech clarity and pronunciation.

Furthermore, the styles of the cognitive exercises generated by the cognitive exercise computation agent 110 can be adjusted dynamically based on the exercise styles 138 stored in the individualized knowledge system 130. For example, if the exercise styles 138 indicate a preference for concrete and focused exercises, the cognitive exercise computation agent 110 can generate exercises that focus on specific, tangible details and facts within a particular domain or subject area. Conversely, if the exercise styles 138 suggest that the user responds better to broad, exploratory, or imaginative exercises, the agent can create exercises that cover a wide range of topics, encourage curiosity and open-ended exploration, or incorporate creative thinking and visualization. Additionally, the exercise styles 138 can inform the cognitive exercise computation agent 110 to design exercises that are personalized, conversational, experiential, or multisensory, tailoring the format and delivery to the user's preferred learning modalities and engagement styles. The cognitive exercise computation agent 110 can also adjust the exercise styles to incorporate game-based, kinesthetic, collaborative, or adaptive elements, introducing elements of play, physical movement, social interaction, or dynamic difficulty adjustment to enhance the user's motivation and engagement. By leveraging the enhancement areas 136 and exercise styles 138 stored in the individualized knowledge system 130, the cognitive exercise computation agent 110 can generate exercises that are not only tailored to the user's specific cognitive needs but also presented in a format and style that aligns with their preferences, learning styles, and therapeutic goals, maximizing the effectiveness and enjoyment of the cognitive training experience.

During a user session, the API 140 presents a user, a cognitive exercise generated by the cognitive exercise computation agent 110 employing the LLM 122 and the NLP tools 124. The user conducts the cognitive exercise and may provide feedback to the cognitive exercise. The user's response and feedback to the cognitive exercise and received by the cognitive exercise computation agent 110, which can be used to update the memory list 134, the enhancement areas 136, and the exercise styles 138. The cognitive exercise computation agent 110 presents a personalized interface at the API 140 based on the user's disabilities, preferences, or limitations can enhance accessibility and case of use, while avoiding overwhelming or distracting elements. Such personalized interface can reduce anxiety reduce anxiety and create a comfortable learning environment. The personalized interface at the API 140 that incorporates personal details and life situations into the interface design, which can foster a sense of empathy and rapport, creating a supportive learning environment.

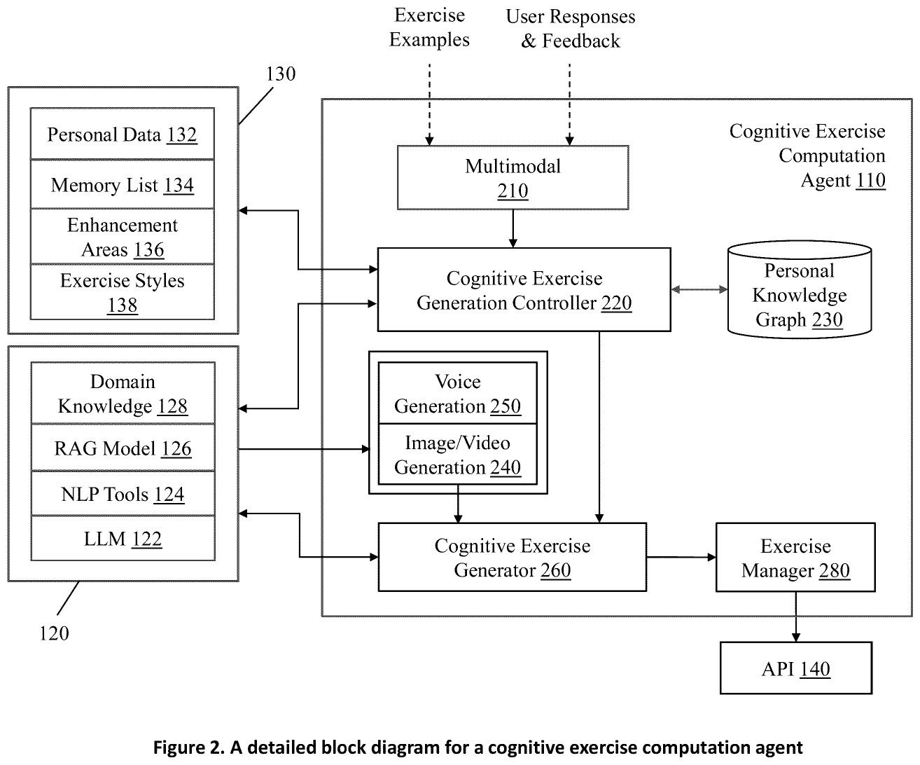

Referring to FIG. 2, the cognitive exercise computation agent 110 includes a multimodal module 210, a cognitive exercise generation controller (CEGC) 220, personal knowledge graph 230, image/video generation module 240, voice generation module 250, a cognitive exercise generator (CEG) 260, and an exercise manager 280.

The multimodal module 210 can receive different forms of inputs from a user such as voice, images, video, text, touch, and so on, and convert them into digital information to input to the cognitive exercise generation controller 220.

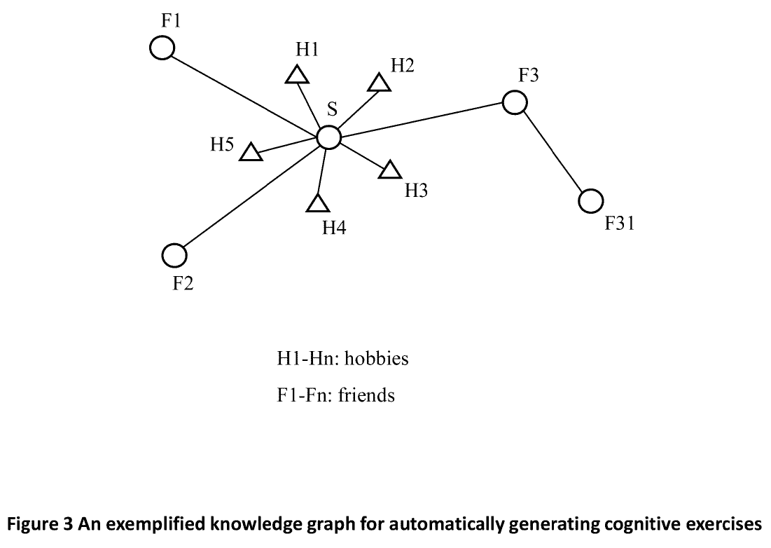

The personal knowledge graph 230 stores individual data points associated with a user and as importantly the relationships between these data points. An exemplified knowledge graph for a user is shown in FIG. 3. The exemplified knowledge graph shows two aspects, or two dimensions: friends and hobbies, in relation to a user “S”. User “S” has hobbies or interests H1-H5, such as, travel, movies, coffee, football, basketball, pickleball, biking, hiking, yoga, cooking, chess. . . . User “S” is friends with persons F1, F2, F3, and F4. F31 is a friend of F3. It should be understood that a knowledge graph can include many other aspects (or dimensions) such as family members, life experiences, education, career, colleagues or collaborators, daily routines, places traveled, favorite songs, favorite movies, history, cultures. . . . It should be noted that the relationship nodes (F1-F4) in the knowledge graph 230 can represent family members like parents, siblings, spouse, children, grandparents, and cousins. These relationships can be further detailed: “S's mother enjoys gardening” or “S's brother is a musician.” The relationship nodes (F1-F4) in the knowledge graph 230 can also represent colleagues, previous employers, mentors, and collaborators in a professional network. These connections allow the cognitive exercise computation agent 110 to generate memory exercises like recalling past work experiences or specific colleagues. The relationship nodes (F1-F4) in the knowledge graph 230 can also represent social groups such as members in clubs, online communities, religious organizations, or volunteer groups. This allows for exercises that target recalling social interactions or specific group activities.

Furthermore, the activities and interests (H1-H5 . . . ) can include categories each associated with one or more subcategories such as “Outdoor Activities” (hiking, biking), “Sports” (football, basketball), “Creative Pursuits” (cooking, chess, etc.). This allows for targeted memory prompts based on categories. The activities and interests (H1-H5 . . . ) can include skill development such as learning a language, playing a musical instrument, or taking a dance class. These nodes and their connections can be used by the cognitive exercise computation agent 110 to generate exercises that focus on remembering specific skills or steps involved in the activity. The activities and interests (H1-H5 . . . ) can include daily routines such as taking medication, exercising, or completing chores can be helpful for memory exercises related to daily routines. The activities and interests (H1-H5 . . . ) can include major life events like graduations, weddings, vacations, or moving to a new home. These nodes and their connections can be used by the cognitive exercise computation agent 110 to generate exercises that target recalling specific details of these events. The activities and interests (H1-H5 . . . ) and in associated subcategory nodes can include sensory details: Associate hobbies and activities with sensory details like the smell of freshly brewed coffee, the feeling of wind while cycling, or the sound of music. This can enhance memory recall by triggering sensory associations, which can further enhance and retain user's memories. The activities and interests (H1-H5 . . . ) can connect activities with nodes representing specific locations like favorite restaurants, parks, or vacation destinations. This can be used for exercises that target recalling details of past visits or planning new ones.

The knowledge represented in the personal knowledge graph 230 can be used in generating content of the personalized cognitive exercises such as divergent or convergent exercises. Relationships and activities stored in the personal knowledge graph 230 can be used in these exercises by the cognitive exercise computation agent 110. For example, if user “S” enjoys playing basketball (as indicated in H1), an exercise is generated to ask “S” to list professional basketball teams (related concept) the user knows. User “S” is friends with F2, who enjoys cooking (F2's interest). An exercise can be generated to ask “S” to name different types of cuisines (related concept) based on their friend's interest. If user S is connected to H2 “Sports”, an exercise is generated to present “S” with a list of various sports and ask “S” to identify the category name of the list (convergence). The cognitive exercise computation agent 110 can generate exercises that provide a more comprehensive list of daily activities (convergent concept set) such as showering, eating breakfast, etc., and ask user “S” to identify the subset of activities in the list that user “S” typically do after waking up (related concept to user's routine).

The cognitive exercise generation controller 220 is the engine of the cognitive exercise computation agent 110: it synthesizes newly received information, computation resources and stored knowledge to generate different types of cognitive exercises most beneficial to a specific user. The types of cognitive exercises can include convergence naming, divergence naming, confrontation naming, filling blanks in a phrase, synonyms and antonyms, and subject-verb-object sentences, and so on. The cognitive exercise generation controller 220 can receive exercise examples and user response and feedback.

The exercise examples and user response and feedback can be in different formats such as voice, text, UI selection, video, image, facial expressions, gestures, postures, movements, etc., which can be processed by the multimodal 210 to generate a unified digital output to the cognitive exercise generation controller 220.

In some embodiments, the cognitive exercise generation controller 220 is responsive to user exercise feedback. For example, if the user does great on confrontation naming, those exercises may be excluded at least for a period of time. Conversely, if a user has improved best via convergent naming, these exercises will be the focus. Moreover, exercise types can be selected based on the enthusiasm of the user. If a user's interest has shown to decline in certain type of exercises based on the user exercise feedback, a new type of exercises may be selected to enable the cognitive exercise generator 260 to generate improved cognitive exercises afresh to the user.

The cognitive exercise generation controller 220 requests the LLM 122 and/or the NLP tools 124 to analyze the above inputs under the steering using the memories stored in the individualized knowledge system 130. If the user's responses and feedback to the cognitive exercises generated indicate that the user has difficulties in coming up with the right answers to the cognitive exercises generated, the cognitive exercise generation controller 220 can incorporate other user feedback to request the cognitive exercise generator 260 to generate improved cognitive exercise. The cognitive exercise generation controller 220 can ensure the data points used in the cognitive exercises to be consistent with the personal knowledge graph 230.

It should be noted that in exercises in which the user is asked to list a few examples in response to a prompt of a general category name, the popular examples within that category can be stored in the memory list 134, or the personal knowledge graph 230, etc. But the pre-stored information may not be complete. For answers that are not pre-stored, a cognitive generation controller 220 can enlist the assistance of the NLP tools 124 and LLM 122 to determine if the user's answers indeed belong to the category names in the prompt of the exercise.

An important characteristic of the disclosed system is that the cognitive generation controller 220 can conduct analysis of the user response and feedback, and then steer cognitive exercise generator 260. The CEGC 220 receives various inputs including user information (preferences, goals), recent cognitive assessment results (if applicable), and real-time context (optional). The CEGC leverages the LLM 122 and NLP tools 124 to analyze the user's individualized knowledge system 130. This analysis can involve one or more of the following steps: identifying relevant memories: the LLM 122 and the NLP tools 124 scan the IKS for memories and experiences connected to the exercise topic or user preferences; understanding memory relationships: the LLM 122 and the NLP tools 124 analyze the relationships between memories, identifying potential connections and themes; gauging emotional associations: the LLM 122 and the NLP tools 124 can analyze user-provided feedback or past exercise responses to understand emotional connections to specific memories; steering the CEG 260: based on the analysis of inputs and memories, the CEGC 220 guides the selection or generation of exercises by the CEG 260. This “steering operation” can include selecting exercise type: The CEGC may choose exercise types like synonym/antonym generation, convergent naming (identifying a category based on examples), or short-term memory recall based on the user's needs and the analyzed memories; tailoring difficulty: the CEGC 220 can inform the CEG 260 of the appropriate difficulty level for the exercise based on user performance and memory analysis; and personalizing Content: The CEGC ensures the CEG utilizes user-specific details or memories from the individualized knowledge system 130 within the exercise content.

The analysis of user responses and feedback by the cognitive generation controller 220 and the steering of the cognitive exercise generator 260 can result in adaptive learning for the user as well as for the computer system 100. The CEGC 220 monitors user responses and feedback to the generated exercises. If the user consistently struggles with the exercises, the CEGC 220 recognizes this as a potential difficulty. The CEGC 220 can then incorporate other user feedback, such as preferred exercise types or areas of interest, to inform the CEG 260 when generating improved exercises. Throughout the process, the CEGC ensures that the data points and themes used in the exercises remain consistent with the data stored in the individualized knowledge system 130, which prevents inconsistencies and reinforces the accuracy of the knowledge graph. Overall, the CEGC 220 acts as a central controller, analyzing user information, memory data, and feedback to steer the exercise generation process. This adaptive approach ensures that the generated exercises are personalized, challenging, and ultimately effective in improving the user's cognitive abilities.

Referring to FIG. 2, the cognitive exercise generator 260 generates the personalized cognitive exercises by employing the LLM 122 and the NLP tools 124 under the control of the individualized knowledge system 130. The personalized cognitive exercises generated can incorporate personal content in the personal data 132. For example, a divergent exercise can ask a user to list places where the user has lived. The personalized cognitive exercises generated can target the subjects stored in the memory list 134 that the user had difficulties in remembering. In the example above, the user may have trouble remembering a city where he lived.

Incorporating familiar words, names, places, and events into the exercises can enhance relatability, making the practice sessions more engaging and meaningful. Additionally, focusing on words and conversational contexts that are directly relevant to the user's daily interactions can increase the practical applicability of the skills learned, facilitating smoother communication in real-world situations.

The personalized cognitive exercises generated can target areas where the user needs to improve, as specified in the enhance areas 136. For example, if a user has difficulty in recognizing, understanding, or pronouncing a phoneme, wording comprising that phoneme can be included repeatedly in the personalized cognitive exercises. Moreover, the styles of the exercises can vary according to the exercise style 138.

The cognitive exercise generator 260 can employ the LLM 122 and the NLP tools 124 to provide additional semantic, phonetic, or orthographic cues. For example, in response to the cues provided, the user may have trouble remembering or speaking all the syllables in the whole word “transportation.” The cognitive exercise generator 260 can generate a display or a pronunciation of the syllable “trans” or “port” as additional cues for the user to produce the answer “transportation”.

The cognitive exercise generation controller 220 requests the image/video generation module 240 for the personalized cognitive exercises generated by the cognitive exercise generator 260. The cognitive exercise generation controller 220 requests the voice generation module 250 to generate voices for the personalized cognitive exercises generated by the cognitive exercise generator 260. The personalized cognitive exercises can be at least in part displayed or played in animation or videos. The LLM 122 and the NLP tools 124 produce the sentences and the right sequence for the voices and image/video sequences in the personalized cognitive exercises. The exercise module 280 can select and output one of the personalized cognitive exercises to present to the user at the API 140.

The CEGC 220 can track user performance data on different types of exercises (e.g., synonym generation vs. memory recall). Statistical analysis of this data can reveal which exercise types are most effective in improving specific cognitive skills for different user groups. This allows the CEGC 220 to prioritize these exercises for future generations. The CEGC 220 can analyze user responses and completion rates for exercises of varying difficulty, the CEGC 220 can determine the optimal difficulty level for each user and exercise type. This ensures the user is challenged appropriately without becoming discouraged. The CEGC 220 can track user performance on memory-based exercises over time. Statistical analysis of this data can assess the effectiveness of the exercises in promoting long-term memory retention of the targeted information. The CEGC 220 can analyze user performance data statistically across demographics, cognitive strengths/weaknesses, and exercise preferences can help personalize exercise generation. The CEGC 220 can use this data to tailor exercises to individual learning styles and needs. By analyzing user responses to specific stimuli within exercises (e.g., images, voices), the CEGC 220 can identify which elements are most engaging and effective. This data can be used to optimize future exercise content for better learning outcomes. Advanced statistical models can be used to predict user performance on certain exercises based on past performance and cognitive assessment data. This allows the CEGC 220 to pre-emptively adjust difficulty levels or exercise types for a more tailored experience.

In one example, given a convergence naming exercise in the cognitive exercise example for “fruit,” a generated cognitive exercise can include “bus, train, and airplane” as cues, with “transportation” to be the answer to the convergence naming. The generated cognitive exercise can include each of the written words along with a spoken representation and an image for each word. The cognitive exercise generator 260 can generate a large number of cognitive exercises in response to one or more exercise examples under the constraints of the personal data 130, the memory list 140 stored in the individualized knowledge system 130. The emphases and style of the cognitive exercises can be influenced by the enhancement areas 136 and exercise styles 138 also stored in the individualized knowledge system 130.

Referring to FIG. 2, an important feature of the presently disclosed system is that the cognitive exercise generation controller 220 can provide a personalized, targeted memory cue to the user. The cognitive exercise generation controller 220 accomplishes this personalization using the personal knowledge graph 230, the RAG model 126, and the domain knowledge database 128. Using a user's favorite sports team as an example, the Golden State Warriors. This includes players' names, statistics, memorable games, championship seasons, fan rituals, etc. When the user is discussing the Warriors but forgets a player's name, like Klay Thompson, the cognitive exercise computation agent 110 passes the context to the RAG model 126, which searches the personal knowledge graph 230 and retrieves helpful, relevant knowledge data points or association thereof about Klay to jog the user's memory. For example, if the user says, “I can't remember the name of that Warriors guard who is an incredible 3-point shooter” the model could respond “Are you thinking of #11, the Splash Brother nicknamed ‘Game 6 Klay’ Thompson?” By focusing on pertinent facts around Klay's position, jersey number, shooting ability and playoff performances, the cognitive exercise generation controller 220 can provide a personalized, targeted memory cue to the user. The knowledge graph in the RAG model 126 limits personalized cognitive exercises to verifiable facts about the Warriors, removing the risk of harmful fabrication. Additionally, training the RAG model 126 directly on this domain-specific graph allows retrieving helpful facts matched to the user's interests and conversation style.

In contrast to traditional generative models that may hallucinate ungrounded outputs, the RAG model 126 is restricted to the personalized cognitive exercises grounded in the user's personalized knowledge graph. This prevents manufacturing of false, illegal, or insensitive content that could be generated by AI systems without proper verification. Additionally, the RAG model 126 has been fine-tuned with techniques such as self-consistency to increase output veracity and self-talk to reduce toxic language. Human-in-the-loop processes allow users to verify accuracy of the exercise content and provide feedback to further improve exercise quality in an iterative, trustworthy process. In this way, the RAG model 126 enables the system 100 to dynamically serve thoughtful, truthful personalized cognitive exercises to match each user's context and history. The hybrid retrieval and generation approach can avoid harmful AI behaviors while assisting users in overcoming inconvenient memory lapses.

On one hand, at the heart of the personalized memory cue generation lies the powerful personal knowledge graph (KG) 230 (FIG. 2). This rich repository of the user's personal information, from favorite sports teams to historical trivia, allows the system to provide incredibly relevant memory prompts. Imagine discussing the Golden State Warriors with a friend and forgetting a player's name. In this scenario, the KG acts as a personalized encyclopedia, readily accessible to the system. The Retrieval-Augmented Generation (RAG) model (126) leverages this KG to pinpoint the missing, inaccurate or context specific information, which needs to be updated time to time.

On the other hand, the personalized memory cue generation relies heavily on the granular detail stored within the user's knowledge graph (KG) (FIG. 2). This KG serves as a factual foundation for the RAG model (126) to operate on. For example, when a user mentions the Golden State Warriors but forgets a player's name, the KG provides a wealth of information-players' names, statistics, memorable games, etc. The RAG model then utilizes this factual data to craft a relevant memory cue, such as “Are you thinking of #11, the Splash Brother nicknamed ‘Game 6 Klay’ Thompson?” This focus on verifiable facts stored within the KG ensures the accuracy and effectiveness of the memory prompts, preventing the generation of false or misleading information.

Referring to FIG. 2, the user's response and feedback to the cognitive exercise are received by the cognitive exercise computation agent 110. This feedback can be analyzed using natural language processing (NLP) techniques and other machine learning algorithms to extract valuable insights. These insights can then be used to dynamically update and refine the user's profile within the individualized knowledge system 130, specifically the memory list 134, the enhancement areas 136, and the exercise styles 138.

For instance, if the user struggles with a particular exercise or provides feedback indicating difficulty with a specific memory type or cognitive skill, the cognitive exercise computation agent 110 can update the memory list 134 to prioritize and focus on strengthening that area. Additionally, the enhancement areas 136 can be adjusted to reflect the need for more exercises targeting the identified weakness or cognitive deficit.

Furthermore, the user's feedback on the exercise style, format, or delivery method can inform updates to the exercise styles 138. If the user expresses a preference for a particular exercise style (e.g., imaginative, exploratory, or conversational) or indicates that a certain style was particularly engaging or effective, the exercise styles 138 can be adjusted to prioritize those preferred formats in future exercise generation.

The cognitive exercise computation agent 110 can also analyze the user's performance data, such as response times, accuracy rates, or patterns of errors, to gain insights into the user's cognitive strengths and weaknesses. This information can be used to refine the memory list 134, enhancement areas 136, and exercise styles 138, ensuring that the cognitive exercises remain tailored and optimized for the user's evolving needs and abilities.

By continuously incorporating user feedback and performance data, the cognitive exercise computation agent 110 can dynamically adapt and personalize the cognitive training experience, providing a responsive and effective approach to cognitive rehabilitation and enhancement.

An important characteristic of the disclosed system extends beyond the technical aspects of generating personalized cognitive exercises. The cognitive exercise generation controller 220 incorporates a unique and vital element—the preservation of kindness, nuanced cultural thoughtfulness, sensibility, and cognizance in moral and civil discourse. In the realm of memory assistance, the cognitive exercise generation controller 220, the personal knowledge graph 230, and the domain knowledge 128 are designed not only to prompt the retrieval of words but to do so with an awareness of the cultural context, emotional sensitivity, and ethical considerations. This ensures that the generated exercises are not only accurate but also align with the user's values, promoting respectful and considerate communication even in challenging situations. The cognitive exercise generation controller 220 and the domain knowledge 128 incorporate mechanisms to avoid generating personalized cognitive exercises that may provoke or offend, contributing to a positive and inclusive conversational experience.

Moreover, the cognitive exercise generation controller 220, the personal knowledge graph 230, and the domain knowledge 128 are also designed to possess thoughtfulness with cultural nuance. Delicate consideration of cultural nuances is integrated into the system, ensuring a thoughtful approach that takes into account the intricacies and subtleties of diverse cultural contexts. This feature goes beyond mere accuracy, emphasizing a respectful understanding of the cultural background of users, thereby enhancing the overall effectiveness and appropriateness of the generated personalized cognitive exercises.

The computer system 100 and the cognitive exercise computation agent 110 incorporate human-like qualities to encourage widespread user acceptance. By humanizing the system, making it relatable and attuned to human values and sensitivities, users are more inclined to engage with and trust the disclosed system and methods. This described approach fosters a user-friendly environment, increasing the likelihood that people will embrace and use the software for their memory assistance needs. For example, different individuals may respond better to certain exercise formats (e.g., visual, auditory, kinesthetic) and can be optimized and tailored to allow individual users to achieve more effective learning.

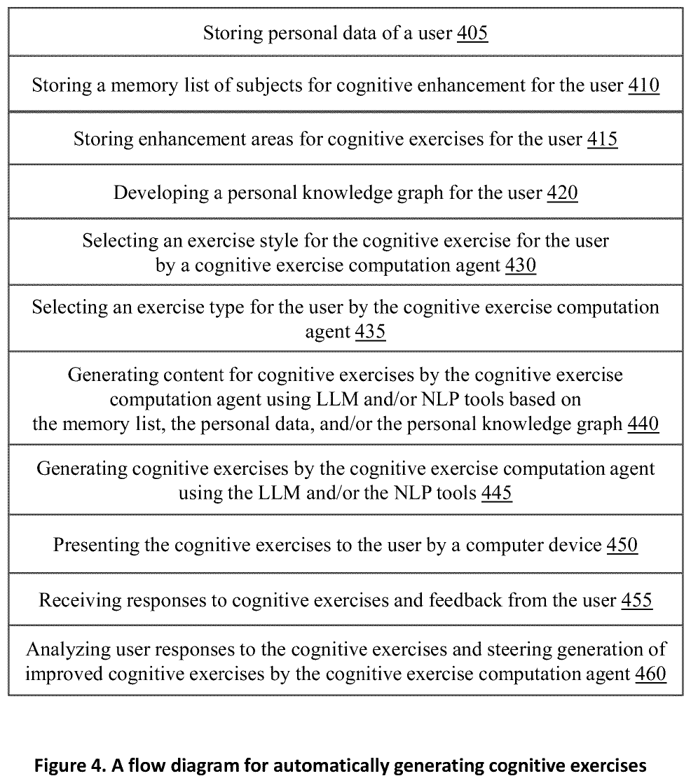

In some embodiments, a computer system for automatically generating cognitive exercises can include one or more of the following steps. Referring to FIG. 4, personal data of a user is stored (step 405) in an individualized knowledge system. The personal data can include a user's important relationships such as family, friends, and caretakers, daily activities, the user's interests and hobbies, the user's life experiences such as upbringing, education and occupations, and travel experiences, places important to the user, notable events, and accomplishments in the user's life, etc. The personal data can include user's unique circumstances, challenges, and life experiences. The personal data can be built up during application initiation process or training sessions and updated during use sessions based on feedback from the user.

Details about personal data of users and its storage and their suage in automatically generating cognitive exercises for users are described above in relation to FIGS. 1-3.

A memory list of subjects for cognitive enhancement for a user is stored (step 410), which can be stored in the individualized knowledge system. The user may have difficulty remembering the subjects in the memory list. The subjects can include daily routine activities, healthcare needs, relationships, words, objects, experiences, events, interests, hobbies, occupational and professional terminologies, or details related to their past or current professions, historical facts, literature, art, music, and various other topics or domains that are relevant and challenging for the specific user's needs. Details about list of words and objects, and associated memory list for users are described above in relation to FIGS. 1-3.

Enhancement areas for cognitive exercises for the user are stored (step 415), which can be stored in the individualized knowledge system. Details about enhancement areas for cognitive enhancement for automatically generating cognitive exercises for users are discussed above in relation to FIGS. 1-3.

Personal knowledge graphs for the user are developed (step 420). Details about knowledge graphs and personal knowledge graphs for automatically generating cognitive exercises for users are discussed above in relation to FIGS. 1-3.

An exercise style is selected for the cognitive exercise for the user by a cognitive exercise computation agent (step 430). An exercise type is selected for the user by the cognitive exercise computation agent (step 435). Details about exercise styles, the impact of exercise style on the effectiveness of the exercises, and the exercise type and exercise style selections for automatically generating cognitive exercises for users are discussed above in relation to FIGS. 1-3.

Content for cognitive exercises is generated by the cognitive exercise computation agent using LLM and/or NLP tools based on the memory list and the personal knowledge graph (step 440). Processes and examples about incorporating content and personalized content in automatically generating cognitive exercises for users are discussed in detail above in relation to FIGS. 1-3.

Cognitive exercises are generated by the cognitive exercise computation agent using LLM and/or NLP tools (step 445). Processes and examples about the cognitive exercise computation agent using LLM and/or NLP tools to automatically generate cognitive exercises for users are discussed in detail above in relation to FIGS. 1-3.

The cognitive exercises are presented to the user by a computer device (step 450). Details about personalized presentation of cognitive exercises are discussed above in relation to FIGS. 1-3.

Responses to cognitive exercises and feedback are received from the user (step 455). Details about responses to cognitive exercises and feedback from the user, and related analysis, and automatically generating improved cognitive exercises for the users are discussed above in relation to FIGS. 1-3.

The user's responses and feedback to the cognitive exercises are analyzed by the cognitive exercise computation agent, which is used by the cognitive exercise computation agent to steer the generation of improved cognitive exercises (step 460). Detailed steps and the analysis of the user's responses and feedback, and details of the resulting steering of the generation of improved cognitive exercises by the cognitive exercise computation agent are disclosed above in relation to FIGS. 1-3.

Only a few examples and implementations are described. Other implementations, variations, modifications and enhancements to the described examples and implementations may be made without deviating from the spirit of the present invention. It should be noted that the presently disclosed computer system for automatically generating cognitive exercises is not limited to the specific examples described above. The presently disclosed computer system can be implemented on a mobile application, a cloud system, a local computer network, and/or a combination thereof. The presently disclosed computer system is compatible with different types of large language models such as foundation models, domain-specific models, zero-shot models, and single-modal or multimodal models. The presently disclosed computer system can also employ different forms of natural language processing tools such as sentiment analysis, named entity recognition, summarization, topic modeling, text classification, keyword extraction, and lemmatization and stemming. The large language models and the natural language processing tools can be implemented on a computer network, a cloud system, or on one or more user devices.

1. A computer system for automatically generating cognitive exercises, comprising:
a computer memory configured to store a memory list comprising subjects that a user has difficulty to remember, wherein the computer memory is configured to store personal data associated with the user; and
one or more computer processors in communication with the computer memory, the one or more computer processors configured to execute a cognitive exercise computation agent and a large language model responsive to the cognitive exercise computation agent,
wherein the cognitive exercise computation agent is configured to retrieve the memory list and the personal data associated with the user from the computer memory,
wherein the cognitive exercise computation agent is configured to produce generated cognitive exercises using the large language model under constraints of the memory list and the personal data associated with the user.

2. The computer system of claim 1, wherein the computer memory is configured to store a personal knowledge graph associated with the user,
wherein the cognitive exercise computation agent is configured to produce the generated cognitive exercises based on the personal knowledge graph using the large language model.

3. The computer system of claim 2, wherein the personal knowledge graph comprises a user node representing the user, one or more first nodes in connection with the user node, representing relationships that the user has, and one or more second nodes in connection with the user node, representing interests and hobbies of the user.

4. The computer system of claim 1, wherein the computer memory is configured to store exercise styles comprising concrete, focused, broad, stimulative, imaginative, exploratory, personalized, conversational, real-life situation, multimedia, multi-sensory, kinesthetic that involves physical movement or gestures, collaborative with other users, adaptive, progressive, or thematic around specific themes, or narratives,
wherein the cognitive exercise computation agent is configured to control the large language model to produce generated cognitive exercises further under constraint of the exercise styles stored in the computer memory.

5. The computer system of claim 1, wherein the cognitive exercise computation agent comprises a cognitive exercise generator configured to produce the generated cognitive exercises using the large language model under constraints of the memory list and the personal data associated with the user.

6. The computer system of claim 5, wherein the cognitive exercise computation agent comprises a cognitive exercise generation controller configured to receive the memory list and the personal data associated with the user, wherein the cognitive exercise generation controller is configured to control the cognitive exercise generator and the large language model to produce the generated cognitive exercises under constraints of the memory list and the personal data associated with the user.

7. The computer system of claim 6, wherein the cognitive exercise generation controller is configured to receive user responses or feedback,
wherein the cognitive exercise generation controller is configured to steer the cognitive exercise generator and the large language model to produce improved cognitive exercises in response to user responses or feedback.

8. The computer system of claim 6, wherein the cognitive exercise generation controller is configured to receive an example cognitive exercise,
wherein the cognitive exercise generator is configured to produce the generated cognitive exercises that mimic the example cognitive exercise.

9. The computer system of claim 5, wherein the cognitive exercise computation agent comprises an image/video generator configured to produce images or videos using the large language model under constraints of the memory list and the personal data associated with the user, wherein the images or videos are incorporated into the generated cognitive exercises.

10. The computer system of claim 5, wherein the cognitive exercise computation agent comprises a voice generation module configured to produce a voice to be incorporated into the generated cognitive exercises.

11. The computer system of claim 1, wherein the cognitive exercise computation agent comprises a multimodal module configured to receive user responses and feedback in one or more forms of voice, images, video, text, or touch.

12. The computer system of claim 1, wherein the memory list includes daily routine activities, healthcare needs, relationships, words, objects, experiences, events, interests, hobbies, occupational and professional terminologies, past experiences, professional activities, historical facts, literature, art, or music.

13. The computer system of claim 1, wherein the computer memory is configured to store a list of enhancement areas in which the user's memory needs to be improved, wherein the cognitive exercise computation agent is configured to produce the generated cognitive exercises using the large language model further in accordance with enhancement areas associated with the user,
wherein the enhancement areas comprise motor-muscular-phonetics exercises, language syllable exercises, language exercises, cognitive exercises including convergence naming, divergence naming, or confrontation naming, daily activities, levels of memory loss, or general memory exercises for brain wellness.

14. A computer system for automatically generating cognitive exercises, comprising:
a computer memory configured to store a memory list comprising subjects that a user has difficulty to remember, wherein the computer memory is configured to store personal data associated with the user in which the user's memory needs to be improved; and
one or more computer processors in communication with the computer memory, the one or more computer processors configured to execute a cognitive exercise computation agent and a large language model responsive to the cognitive exercise computation agent,
wherein the cognitive exercise computation agent comprises a cognitive exercise generation controller configured to retrieve the memory list and the personal data associated with the user from the computer memory,
wherein the cognitive exercise computation agent comprises a cognitive exercise generator,
wherein the cognitive exercise generation controller is configured to control the cognitive exercise generator and the large language model to produce the generated cognitive exercises under constraints of the memory list and the personal data associated with the user,
wherein the cognitive exercise generation controller is configured to receive user responses or feedback, wherein the cognitive exercise generation controller is configured to steer the cognitive exercise generator and the large language model to produce improved cognitive exercises in response to user responses or feedback.

15. The computer system of claim 14, wherein the computer memory is configured to store a personal knowledge graph associated with the user,
wherein the cognitive exercise computation agent is configured to control the large language model to produce improved cognitive exercises based on the personal knowledge graph.

16. The computer system of claim 15, wherein the personal knowledge graph comprises a user node representing the user, one or more first nodes in connection with the user node, representing relationships that the user has, and one or more second nodes in connection with the user node, representing interests and hobbies of the user.

17. The computer system of claim 14, wherein the computer memory is configured to store exercise styles comprising concrete, focused, broad, stimulative, imaginative, exploratory, personalized, conversational, real-life situation, multimedia, multi-sensory, kinesthetic that involves physical movement or gestures, collaborative with other users, adaptive, progressive, and thematic around specific themes, or narratives,
wherein the cognitive exercise computation agent is configured to control the large language model to produce generated cognitive exercises further under constraint of the exercise styles stored in the computer memory.

18. A computer-implemented method for automatically generating cognitive exercises, comprising,
storing personal data of a user in a computer memory;
storing a memory list of subjects for cognitive enhancement for the user in the computer memory;
generating content for cognitive exercises by a cognitive exercise computation agent, executed by one or more computer processors, using LLM and/or NLP tools based on the personal data and the memory list stored in the computer memory;
generating cognitive exercises by the cognitive exercise computation agent using the LLM and/or the NLP tools; and
presenting the cognitive exercises to the user by a computer device.

19. The computer-implemented method of claim 18, further comprising:
developing a personal knowledge graph for the user; and
generating the content for cognitive exercises by the cognitive exercise computation agent using LLM and/or NLP tools further based on the personal knowledge graph.

20. The computer-implemented method of claim 18, further comprising:
selecting an exercise style for the user by the cognitive exercise computation agent;
selecting an exercise type for the user by the cognitive exercise computation agent; and
generating the cognitive exercises of the selected exercise type in the selected exercise style using LLM and/or NLP tools by the cognitive exercise computation agent.

21. The computer-implemented method of claim 18, further comprising:
receiving responses to cognitive exercises and feedback from the user;
analyzing the responses and the feedback to the cognitive exercises; and
steering generation of improved cognitive exercises by the cognitive exercise computation agent.
